(19) United States
(12) Patent Application Publication
(10) Pub. No.: US 2026/0077213 A1
(43) Pub. Date: Mar. 19, 2026

(54) METHOD FOR DETERMINING A TREATMENT PLAN FOR A RADIOTHERAPY TREATMENT

(71) Applicant: Brainlab SE, Munich (DE)

(72) Inventors: Cornelis Kamerling, Munich (DE); Merle Schlottmann, Munich (DE); Stephan Zöllinger, Munich (DE); Adrian Fochi, Munich (DE); Mattia Donzelli, Munich (DE)

(21) Appl. No.: 19/110,061

(22) PCT Filed: Sep. 8, 2022
(86) PCT No.: PCT/EP2022/074992

§ 371 Date: Mar. 8, 2025

(51) Int. Cl. A61N 5/10

(52) U.S. Cl. CPC A61N 5/1031

(57) ABSTRACT

Disclosed is a computer-implemented method for determining a treatment plan for a radiotherapy treatment including a dose distribution, the method comprising the steps of (a) determining a plurality of clinical goals, the plurality of clinical goals associated with a dose distribution, (b) determining, based at least in part on a user input, a priority for each of the clinical goals, (c) automatically determining a subset and order of scripts among a plurality of scripts, based on the plurality of clinical goals and their respective priority, each script configured to, when executed, adapt one or more optimization objectives and/or adapt weightings for optimization and/or provide and/or adapt an optimization structure, (d) generating a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization,

### FIELD OF THE INVENTION

The present invention relates to a computer-implemented method for determining a treatment plan including a dose distribution, a processing system, a computer program product, and a computer-readable medium.

### TECHNICAL BACKGROUND

Irradiation with ionizing radiation, for example in the course of radiotherapy treatment, generally presupposes that planning is performed in advance as to how the irradiation is performed. This may be referred to as radiotherapy planning.

The planning may result in a treatment plan, which may, among others, comprise a spatial dose distribution.

In general, a treatment plan and dose distributions may be determined by applying an optimization method.

In conventional radiotherapy treatment planning, treatment planners are tasked to ensure that certain clinical goals, often provided by clinical professionals, are fulfilled. They can make use of conventional radiotherapy treatment planning methods. Examples for these methods are manually setting and adapting of weightings and/or constraints, and/or manual generation of optimization structures and/or objectives.

To achieve clinical goals, sometimes complex manual changes to the global optimization problem have to be made. These changes allow to steer the optimization algorithm in a certain direction, for example by formulating local clues on where to constrain or loosen the problem locally. This already, as such, is difficult and involves significant trial-and-error.

Moreover, conventional optimization algorithms work with trade-offs, such that changing parameters to achieve one goal will usually interfere with the achievement of another goal. This may in some cases not be acceptable and, accordingly, leads to even more trial and error and/or complex user interaction being required to obtain acceptable results.

As a result, inacceptable delays my occur, and the method is cumbersome and error-prone.

The present invention has the object of providing a method, processing system, computer program product, and computer-readable medium that allow for overcoming at least some of the above-identified challenges.

The invention can be used for providing information that may be applicable to procedures e.g. in connection with a system for image-guided radiotherapy such as VERO® and ExacTrac®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention provides a method, a processing system, a computer program product, and a computer-readable medium according to the independent claims. Preferred embodiments are laid down in the dependent claims.

The present disclosure provides, among others, a computer-implemented method for determining a treatment plan for a radiotherapy treatment including a dose distribution, the method comprising the steps of (a) determining a plurality of clinical goals, the plurality of clinical goals associated with a dose distribution, (b) determining, based at least in part on a user input, a priority for each of the clinical goals, (c) automatically determining a subset and order of scripts among a plurality of scripts, based on the plurality of clinical goals and their respective priority, each script configured to, when executed, adapt one or more optimization objectives and/or adapt weightings for optimization and/or provide and/or adapt an optimization structure, (d) generating a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

### GENERAL DESCRIPTION OF THE INVENTION

In this section, a description of the general features of the present invention is given for example by providing possible embodiments of the invention.

The present disclosure provides computer-implemented method for determining a treatment plan for a radiotherapy treatment including a dose distribution, the method comprising the steps of (a) determining a plurality of clinical goals, the plurality of clinical goals associated with a dose distribution, (b) determining, based at least in part on a user input, a priority for each of the clinical goals, (c) automatically determining a subset and order of scripts among a plurality of scripts, based on the plurality of clinical goals and their respective priority, each script configured to, when executed, adapt one or more optimization objectives and/or adapt weightings for optimization and/or provide and/or adapt an optimization structure, (d) generating a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

In other words, the present disclosure allows for automatically determining, without requiring any user intervention going beyond user input that is used for determining the priority of the clinical goals, a dose distribution that is part of a radiotherapy treatment plan. To do so, the available information, i.e., the clinical goals and their priorities, are used as input to automatically select a plurality of scripts and automatically determine an order in which to perform the scripts. The scripts are executed in this order, thereby successively creating and/or adapting optimization structures, weightings, and/or objectives. The creating and/or adapting performed by executing each of the scripts aims at meeting a clinical goal for which the script was selected.

A dose distribution may be yielded by each of the scripts and said dose distribution may be adapted by the subsequent script. When the execution of the scripts is terminated, for example after execution of the last script of the subset of scripts or when an abortion is triggered, the results of the script that was executed last may be the final results. For example, the dose distribution may be the final dose distribution and/or the optimization structure(s) and/or weightings, and/or objectives may be the final optimization structure(s), weightings, and objectives, respectively.

The method of the present disclosure, thus, allows for significantly reducing trial-and-error. If a solution to the problem exists, the method will efficiently and reliably yield said solution. In addition, due to the modular approach of separate prioritized goals and scripts selected accordingly, the method provides a basis for avoiding trade-offs that may not be necessary or desirable and/or for avoiding that higher-ranking goals are violated when trying to meet lower-ranking goals. One example making use of this advantage is explained in detail further below, wherein certain results can be locked.

The method of the present disclosure allows for scripted generation and/or adaption of optimization structures, optimization objectives, and/or weightings for optimizations (also referred to as optimization weightings or weightings herein) that allow for achieving a prioritized list of clinical goals in an iterative manner that does not require user interaction. The method is particularly relevant for scenarios where “in-between” solutions as often obtained with the existing methods due to trade-offs are not acceptable, as it provides an automated manner of exploring solutions not falling in the “in-between” regions, e.g., allow for exploring the more extreme regions in spite of having to make certain trade-offs.

Thus, the present disclosure provides a method that addresses at least some of the above-described challenges.

According to the present disclosure, a treatment plan for a radiotherapy treatment entails a dose distribution. The term dose distribution may refer to a spatial dose distribution. The dose distribution may, for example, represent a shape of a dose gradient.

The treatment plan may be stored, e.g., for use as a reference for setting operating parameters for operation of a radiation beam source.

The plurality of clinical goals are associated with the dose distribution. For example, the clinical goals may represent goals to be met by the dose distribution to be determined as part of determining the treatment plan. For example, clinical goals may refer to attributes of a dose or dose distribution, for example target values and/or upper and/or lower thresholds of a dose in one or more regions and/or a dose gradient in or between regions. Examples for clinical goals associated with the dose distribution will be provided further below.

The determination of the clinical goals may in part be based on user input, e.g., a user may define and/or select a clinical goal. Alternatively or in addition, clinical goals may be determined based on other sources. Said other sources may comprise a data storage, wherein clinical goals may be stored and, for example, may be associated with certain types radiotherapy treatments, e.g., for irradiation of a specific anatomical structure. The determining of a clinical goal may then entail automatically determining, for the type of radiotherapy treatment plan at hand, clinical goals associated with this type.

The priority of the clinical goals, according to the present disclosure, is determined at least in part based on a user input. As an example, a user may interact with a user interface to set and/or change, i.e., increase or decrease, a priority for one or more clinical goals, e.g., change the priority from a previous or default setting. Optionally, in addition to user input, other factors may be taken into account for setting the priority of the clinical goals. For example, for certain types of radiotherapy treatments there may be default settings or previous settings for priorities of clinical goals, which may be retrieved and used unless otherwise specified by the user input.

The method may optionally comprise that user input setting and/or changing a priority overrules any default or previous settings. Optionally, some priorities may be excluded from this principle, e.g., the setting and/or changing by the user may only be admissible within predetermined ranges or not at all for said excluded priorities.

Determining the priority at least in part based on the user input may take the form of a user indicating an order of the goals in terms of their priorities or, in other words, a priority-based ranking of the goals. Alternatively or in addition to providing an order of priorities, the user might also select a category for each of the goals indicating their priority. Examples might be categories like “may not be violated”, “very high”, “high”, “medium”, “low”, “very low”. Optionally, some prioritization may also be performed automatically and then be open to review and potential editing by the user.

As mentioned above, according to the present disclosure, a subset and order of scripts is automatically determined among a plurality of scripts, based on the plurality of clinical goals and their respective priority.

The term “subset of scripts” refers to two or more scripts selected from a set of scripts comprising two or more, in particular three or more, scripts. The order of scripts refers to the order in which the scripts are to be executed.

The scripts and order of the scripts may be determined such that goals are achieved in order of priority in descending order, i.e., with the highest priority goal being achieved first.

In one example, the automatic determination may include an automatic look-up of a subset and order of scripts associated with the plurality of clinical goals and their priorities. Alternatively or in addition, a rule-based selection of the subset of scripts and their order may be performed. How the automatic determination is suitably made may depend on the specific case at hand, e.g., the number of possible scenarios to be addressed by the automatic determination. Examples will be provided further below.

The term script is to be interpreted broadly. It may refer to an order of instructions to define a low-level optimization problem. In particular, it may define and/or generate optimization structures, dose objectives, volume objectives and/or respective weights. These may be subject to iterative changes during a script run. For example, during a script run, an optimization volume, e.g., a helper volume around a target volume, may be expanded until a given criteria is fulfilled.

As explained above, each script is configured to, when executed, adapt one or more optimization objectives and/or adapt weightings for optimization and/or provide and/or adapt an optimization structure.

Creating and/or adapting an optimization structure may entail creating and/or adapting one or more optimization structures, e.g., a main target region, a transition region adjacent to the main target region, a region associated with a risk structure, and/or a transition region adjacent to the region associated with the risk structure, or the like. An optimization structure, according to the present disclosure, particularly the final optimization structure, similarly, may also comprise one or more optimization structures, e.g., a main target region, a transition region adjacent to the main target region, a region associated with a risk structure, and/or a transition region adjacent to the region associated with the risk structure, or the like.

Adapting objectives may entail changing an objective value. For example, an objective value may be a target dose value, an upper or lower threshold for a dose value, or the like.

Adapting weightings may entail that the weights and/or penalizations in an optimization function are changed. The weightings are used to balance objectives and trade-offs. For example, weightings may be used to define penalization of deviations from objectives.

As mentioned above, the method of the present disclosure comprises generating a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

That is, for example, optimization structures and/or optimization objectives, and/or weightings for optimization may be iteratively adapted by executing the scripts. Accordingly, the dose distribution may also be adapted iteratively by performing an optimization based on the iteratively adapted optimization structures, objectives and/or weightings.

Performing an optimization will yield a dose distribution. Accordingly, a dose distribution is obtained for each script being executed.

All dose distributions except for the dose distribution obtained by the last executed script, which is referred to as final dose distribution in the present disclosure, are considered to be intermediate dose distributions.

In the present disclosure, unless otherwise specified, a script being an “executed script” refers to a script having been successfully executed.

The treatment plan may comprise, particularly only, the final dose distribution. That is, intermediate dose distribution may be omitted from the treatment plan. In case the final dose distribution is obtained after execution of all scripts of the subset if scripts, i.e., after all scripts have been executed successfully, it may be automatically included in the treatment plan.

In case the final dose distribution was obtained prior to execution of all scripts of the subset of scripts, i.e., in case the determining of the dose distribution was terminated or aborted before all scripts have been executed, a user may first be prompted to confirm including the final dose distribution in the treatment plan. To that end, information pertaining to aborting/terminating the determining of the dose distribution may be output to the user. More details concerning aborting the determining will be provided below.

As indicated above, once all scripts have (successfully) executed or the execution of the scripts is aborted, a final dose distribution may be obtained, generated by means of performing the optimization with final optimization structures, objectives, and weightings obtained by the execution of the last executed script.

The term “final”, in the context of the final optimization structure, final optimization objectives, final weightings, and final dose distribution may be used to refer to the optimization structure, objectives, weightings, and dose distribution as determined by successful execution of all of the scripts of the subset of scripts or by execution of the last script performed successfully prior to an abortion of execution.

In terms of the final optimization structure, this may entail one or more optimization sub-structures, e.g., a final main target region and/or one or more final helper structures, e.g., helper volumes.

Accordingly, in particular, executing all scripts of the subset of scripts successfully may result in a final optimization structure, final optimization objectives, and final weightings, and in a final dose distribution, which may be obtained by optimization using the final optimization structure, objectives, and weightings.

At least the final dose distribution and optionally information pertaining to the final optimization structure, final optimization objectives, and final weightings, may be included in the treatment plan. That is, the treatment plan may include at least the final dose distribution.

As seen above, the optimization is based on the optimization structure and/or optimization objectives and/or weightings. This may entail using the optimization structure and/or optimization objectives and/or weightings as input for an optimization algorithm.

The optimization of the present disclosure—as such and itself—may be any optimization method known in the field for automatically determining a dose distribution. For example, it may utilize any known optimization algorithm known in this field.

According to the present disclosure, at least part of an optimization objective and/or a weighting for optimization and/or an optimization structure yielded by executing a script or a sequence of scripts may be locked, such that the locked part cannot be modified by execution of subsequent scripts.

In other words, some results obtained in the course of carrying out the method, particularly, some results obtained by executing the scripts, may be locked. The locked results can then not be changed anymore by subsequent steps. Specifically, the results yielded by executing one or more scripts may be locked such that the clinical goal associated with said one or more scripts cannot be violated in the subsequent steps, e.g., subsequent steps aimed at meeting lower priority goals.

The scripts and order of the scripts may be determined such that goals are achieved in order of priority in descending order, i.e., with the highest priority goal being achieved first. When locking results as described above, this allows specifically for protecting already achieved goals from being affected negatively by subsequent steps.

Locking results may have the advantage that in subsequent steps certain parameters can be excluded from certain trade-offs or only permitted to be subject to trade-offs to a limited degree in subsequent steps. As such, some overall optimization results are accessible that would otherwise not be achieved due to trade-offs made in the course of an optimization. Since the claims provide execution of a specific set of scripts in a specific order, which in turn is determined based on the priority of clinical goals, this means that it is possible to steer indirectly where trade-offs are acceptable or not.

According to the present disclosure, upon determining that a dose distribution cannot be determined without changing a locked optimization objective and/or a locked weighting for optimization and/or a locked optimization structure, e.g., locked from the beginning or after executing a script, the method may abort the generating of the dose distribution and optionally may initiate informing a user of a failure to determine a dose distribution and/or of potential reasons for and/or solutions to a failure to determine a dose distribution. It is noted that the failure to determine a dose distribution, in the present disclosure, is to be understood, in particular, to be a failure to determine a dose distribution meeting all requirements, e.g., clinical goals, restraints, or the like.

As explained above, locking of results may prevent the method from making certain trade-offs. While this ensures that the higher ranked clinical goals are not violated, it may also sometimes lead to situations where no solution can be found, i.e., no distribution meeting all requirements can be determined. Rather than making trade-offs in response to this, the generating of the dose distribution may be aborted. Thus, no dose distribution that violates goals in some untransparent manner is yielded by the method. A user may be informed of the failure and optionally potential reasons for and/or solutions to the failure. In response, for example, a user may make changes to user-defined input parameters, e.g., the clinical goals and/or priorities. Alternatively, rather than aborting the generating of the dose distribution, the generating of the dose distribution may continue despite a violation, optionally after confirmation by a user. For example, there may be rules for determining cases in which continuing despite a violation may be admissible and/or rules for determining cases in which continuing despite a violation requires user confirmation. In any case, information on the violation may be stored, e.g., for analysis, review, and/or decisions in terms of usability of the results.

According to the present disclosure, each script may be executed constrained by at least part of the results of previously executed scripts. In particular, an optimization performed when executing the script may be constrained by the at least part of the results of previously performed scripts.

That is, the method may entail using or carrying over results from previous scripts as constraints for subsequently carried out scripts. This is one example for at least partially locking results.

According to the present disclosure, the generating of the dose distribution may be an iterative process comprising at least a first level of iterations, wherein each iteration is associated with exactly one of the clinical goals and the iterations are performed in the order of the priority of the clinical goals.

That is, the method may proceed in an iterative manner from the highest priority clinical goal to the lowest priority clinical goal, each iteration aiming at meeting the clinical goal it is associated with, such that iteratively the clinical goals are met. When the above-described locking is performed, for example, the method may comprise locking at least some of the results from one iteration and then proceed to the next iteration, which may entail, for example, carrying over some of the locked results as constraints.

These iterations of the first level of iterations may also be seen as highest-level iterations.

According to the present disclosure, the iterative process may comprise a second level of iterations associated with the scripts, wherein each iteration of the second level of iterations is associated with exactly one of the scripts, wherein each iteration of the first level of iterations may be associated with one or more iterations of the second level of iterations.

That is, on a level lower than the first level of iterations, a second level of iterations may be provided, wherein one iteration of the first level may comprise one or more iterations of the second level. For example, when an iteration of the first level is associated with a goal that is associated with multiple scripts, the first level of iterations may be associated with a number of iterations of the second level that corresponds to the number of the multiple scripts.

The iterations of the second level of iterations may be seen as mid-level iterations.

According to the present disclosure, each iteration of the second level of iterations may comprise performing an optimization process or a sequence of optimization processes.

That is, on a level lower than the second level of iterations, a third level of iterations may be provided, wherein one iteration of the second level of iterations may comprise one or more iterations of the third level. An iteration on the third level may be associated with a script or a step linking scripts together.

The iterations of the third level of iterations may be seen as low-level iterations.

The levels of iterations are also referred to as iteration levels in the present disclosure.

The above-described iterative scheme, particularly on multiple levels, allows for accommodating for a wide range of potential scenarios, e.g., combination and prioritization of clinical goals. Due to the iterative scheme, a modular approach can be taken for achieving clinical goals. In case new scripts become available or existing scripts are modified, due to the iterative and modular nature of the method, they can be easily incorporated. Thus, the method allows for increased flexibility in terms of the type of problem that can be solved and in terms of modifying and improving the method steps performed to provide a treatment plan.

According to the present disclosure, the generating of the dose distribution may be a hierarchical process, in particular, wherein each iteration of the first level of iterations may rank higher than the subsequent iterations of the first level of iterations and/or wherein, among the iterations of the second level of iterations associated with one of the iterations of the first level of iterations, each iteration may rank higher than the subsequent iterations.

A hierarchical process may be a process in which some steps and/or results rank higher than other steps and/or results. For example, some results may be immutable, as described above in the context of locking results. The hierarchy may be derived from the priorities of the clinical goals.

The hierarchical process may, in particular, be implemented in the above-described iteration scheme. Within each level of iterations, the rank of the iterations may decrease from each iteration to the next, i.e., the first iteration ranks highest, the last iteration ranks lowest.

As an example, this may allow for ensuring that some trade-offs can be avoided if they would violate higher ranking goals.

According to the present disclosure, the clinical goals may comprise at least one of high dose coverage of a main target region for irradiation, steep dose gradient towards regions adjacent to a/the main target region for irradiation, low upper dose limits in regions adjacent to a/the main target region for irradiation, high lower dose limits in a/the main target region for irradiation, high dose homogeneity, particularly in a/the main target region for irradiation, dose upper limits for a region defined as risk structure, e.g., at least part of an organ like the spinal cord.

The main target region for irradiation may be the region that is intended to receive irradiation, e.g., for treatment. Regions adjacent to said region may, for example, partially or complete enclose the main target region.

The goal of a high dose coverage of the main target region may entail that it is an optimization goal to increase dose coverage as much as possible. Alternatively or in addition, it may entail that the coverage exceeds a predetermined relative or absolute threshold value. This goal may be given a high priority, for example, when it is important that the entire area of the main target region requires adequate irradiation.

The goal of a steep dose gradient towards regions adjacent to the main target region may entail that it is an optimization goal to increase the slope of the dose gradient as much as possible. Alternatively or in addition, it may entail that the slope exceeds a predetermined threshold value. This goal may be given a high priority, for example, when it is important that areas surrounding the main target region receive only little irradiation. For example, where sensitive anatomical structures are located adjacent to the main target area, it may be important that dose falls of steeply towards these structures.

Similarly, the goal of low upper dose limits in regions adjacent to a/the main target region for irradiation may be given a high priority, for example, when it is important that said areas do not receive too much irradiation, e.g., sensitive anatomical structures.

Any goals related to regions adjacent to the main target region may also be given high priority in case this serves to create an optimization structure, which may also comprise a helper structure, as will be explained in detail below.

The goal of high lower dose limits in a/the main target region for irradiation may be given a high priority, for example, when it is important that the main target region does not receive too little irradiation.

The goal of high dose homogeneity, particularly in a/the main target region for irradiation, may be given a high priority, for example, when it is important that there are no hot spots and/or no under-irradiated spots.

The goal of dose upper limits for a region defined as risk structure, e.g., at least part of an organ like the spinal cord, may be given high priority when excessive irradiation of said risk structure may lead to significant damages to said risk structure. The risk structure may or may not be in a region adjacent to the main target region.

According to the present disclosure, the optimization objectives may comprise one or more constraints, in particular, for one or more regions, at least one of a smallest allowed dose value, a highest allowed dose value, a lowest allowed dose gradient, a lowest allowed dose homogeneity value, a highest allowed temporal dose fluctuation value, a target dose value, a target dose gradient value, a target dose homogeneity value, a target spatial dose fluctuation value, a target temporal dose fluctuation value.

It is noted that the dose gradient, dose homogeneity, and temporal dose fluctuation may be quantified in any manner suitable for the application at hand.

As an example, a quantification of homogeneity can be done by evaluating dose/volume points (DVH points) corresponding to the target volume. Such points may correspond to or quantify minimum and maximum dose to the target volume. A quotient of these (e.g. normalized to prescription dose) may be used to quantify homogeneity using a single number.

Dose gradients etc. may be quantified by dose/volume valuation of artificial volumes around the target volume. For example, the volume receiving half of the prescription dose can be quantified. Alternatively, DVH points can be computed for a ring-structure, e.g., encompassing the target volume, of a predetermined size. Alternatively, the distance between dose isolines may be used for quantifying a dose gradient.

According to the present disclosure, the optimization structure may comprise at least a/the main target region for irradiation and may optionally comprise helper structures, e.g. around the main target region, wherein irradiation is not required but may be permitted to meet optimization objectives, and/or helper structures defining and/or surrounding a/the region defined as risk structure, e.g., at least part of an organ like the spinal cord.

As an example, a helper structure wherein irradiation is not required but may be permitted to meet optimization objectives may comprise a transition region having one or more shells around the main target region. Within these shells, constraints may be between constraints in the respective adjacent regions, such that, for example, they allow for steering the dose fall-off at the edges of the main target region. For example, such helper structures may allow to meet any constraints (e.g., lower dose limits and homogeneity requirements) in the main target region without infringing constraints of adjacent regions, e.g., low upper dose constraints.

As another example, helper structures defining and/or surrounding a/the region defined as risk structure, e.g., at least part of an organ like the spinal cord, may be provided to allow for more lenient and, thus, easier to meet, constraints in most regions outside of the main target region, while at the same time protecting critical regions from irradiation. Similarly to the main target region and shell-like helper structures described above, the helper structures defining the region defined as risk structure may also comprise a main region that is surrounded by shells, e.g., shells with more lenient constraints than the main region.

According to the present disclosure, execution of scripts may comprise at least one of adapting a/the main target region for irradiation, creating helper structures, for example helper volumes, around a/the main target region for irradiation, adapting helper structures, for example helper volumes, around a/the main target region for irradiation, adapting constraints within a/the main target region for irradiation and/or within helper structures, for example helper volumes, around a/the main target region for irradiation, and/or creating and/or adapting helper structures defining a/the region defined as risk structure, e.g., at least part of an organ like the spinal cord, and/or adapting constraints within helper structures defining a/the region defined as risk structure, e.g., at least part of an organ like the spinal cord.

Adapting the main target region for irradiation may, for example, entail that the size and/or shape of the main target region is adapted. The adapting may be limited, e.g., by respective constraints.

Creating helper structures may, for example, entail that a size, shape, and position of a helper structure and optionally constraints to be met by the dose distribution within said helper structure are defined. Adapting a helper structure may entail that the size, shape and/or position of the helper structure are changed.

As an example, a script may be provided that determines that a transition region is to be created, the transition region comprising one or more shells and being adjacent to and at least partially surrounding the main target region, the script may further determine a shell-thickness for each of the shells and dose constraints in each shell.

Similarly, a script may be provided that creates an optimization structure representative of a risk structure and optionally an optimization structure in the form of a transition region around the risk structure.

As another example, a script may be provided that creates an adapted main target region having an adapted shape that takes into account a risk structure, e.g., organ at risk.

As yet another example, a script may be provided that increases the size of the main target region, e.g., a main target volume, in order to increase homogeneity of a dose distribution. First, a plan optimization may be run for the main target volume. If the dose is too heterogeneous, the script may apply a ring volume around the target volume and set a dose / objective to this ring, slightly lower than the prescription dose for the target volume.

The script may then run a plan optimization, including the newly added ring (larger volume typically yields more homogeneous dose, especially for small target volumes). This could also be performed iteratively.

According to the present disclosure, steps a) to d) outlined above may not require user intervention.

That is, the determination of the treatment plan may be fully automatic at least once the priorities have been set. Of course, this does not preclude optional user intervention that is performed to adapt/adjust clinical goals and/or priorities in response to the above-described steps of aborting of generating of the dose distribution and informing the user of the failure to determine a dose distribution.

Optionally, all of the steps of the method of the present disclosure may be performed by a data processing system, in particular, fully automatically. In particular, predetermined values may be accessed automatically, even though they may have been entered by a user at some point.

The invention also provides a data processing system configured to carry out the method of the present disclosure, that is one or more, in particular all of the steps of the method of the present disclosure. In particular, the data processing system may comprise one or more processors configured to perform one or more, in particular all of the steps of the method of the present disclosure.

The invention also provides a computer program product comprising instructions which, when the program is executed by a computer, cause the computer to carry out the method of the present disclosure, that is one or more, in particular all of the steps of the method of the present disclosure.

The invention also provides a computer-readable medium comprising instructions which, when executed by a computer, cause the computer to carry out the method of the present disclosure, that is one or more, in particular all of the steps of the method of the present disclosure.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to providing output that may be used for determining settings for operating machine providing a beam, the output and/or settings allowing for meeting given spatial dose constraints. For this reason alone, no surgical or therapeutic activity and in particular no surgical or therapeutic step is necessitated or implied by carrying out the invention.

The features and advantages outlined above in the context of the method similarly apply to the processing system, the computer program product, and the computer readable medium of the present disclosure.

### DEFINITIONS

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Treatment Beam

The present disclosure relates to the field of determining a dose distribution for planning in what manner radiation is to be delivered by a treatment beam. The treatment beam treats body parts which are to be treated and which are referred to in the following as “treatment body parts”. These body parts are for example parts of a patient's body, i.e. anatomical body parts.

The present disclosure relates to the field of medicine and for example to aspects related to beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages: http://www.elekta.com/healthcare_us_elekta_vmat. php and http://www.varian.com/us/oncology/treatments/treatment techniques/rapidarc.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 schematically illustrates a method according to the present disclosure;

FIG. 2 schematically illustrates the iterative and hierarchical nature of a method according to the present disclosure;

FIG. 3 schematically illustrates a method according to the present disclosure;

FIGS. 4a and 4b schematically illustrates example scripts according to the present disclosure; and

FIG. 5 is a schematic illustration of a system according to the present disclosure.

### DESCRIPTION OF EMBODIMENTS

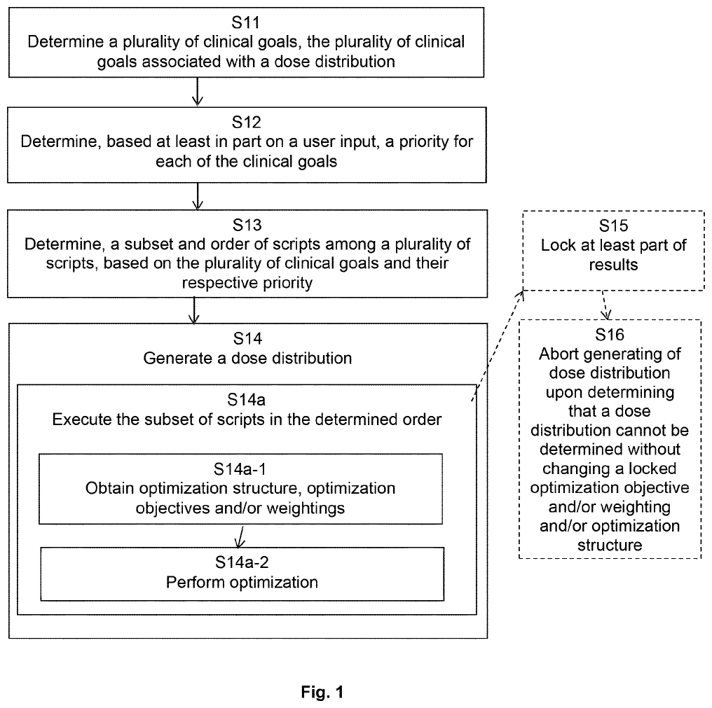

FIG. 1 illustrates exemplary steps of a computer-implemented method for determining a treatment plan for a radiotherapy treatment including a dose distribution according to the present disclosure.

In step S11, a plurality of clinical goals is determined.

As an example, one or more clinical goals may be determined, e.g., selected, based on a user input indicating the clinical goals. Alternatively or in addition, one or more clinical goals may be retrieved automatically, e.g., depending on the radiotherapy application currently at hand. It is to be understood that other means of determining clinical goals are also conceivable.

The plurality of clinical goals are associated with a dose distribution, i.e., they represent goals to be met by the dose distribution to be determined as part of determining the treatment plan. For example, clinical goals may refer to attributes of a dose or dose distribution, for example target values and/or upper and/or lower thresholds of a dose in one or more regions and/or a dose gradient in or between regions.

Clinical goals will ultimately be represented in an optimization problem to be solved to obtain the dose distribution. This will become apparent from the subsequent steps.

In step S12, based at least in part on a user input, a priority for each of the clinical goals is determined. For example, a user may indicate, for the goals determined in step S11 their respective priority. This may simply take the form of indicating an order of the goals in terms of their priorities or, in other words, a priority-based ranking of the goals. Alternatively or in addition to providing an order of priorities, the user might also select a category for each of the goals indicating their priority. Examples might be categories like “may not be violated”, “very high”, “high”, “medium”, “low”, “very low”. Optionally, some prioritization may also be performed automatically and then be open to review and potential editing by the user.

Thus, in step S12, user input, e.g., provided at some point by a clinician and/or a technical expert, can be used for obtaining detailed instructions concerning properties of the dose distribution considered to be relevant in the case at hand. For example, when the clinician views a very steep dose fall-off outside of the main target region for irradiation to be particularly important in the case at hand, e.g., to keep the irradiated region very narrow, then they may give high priority to the goal of maximizing dose fall-off, for example, higher than the goal of high dose coverage up to the edges of the main target region.

In step S13, a subset and order of scripts among a plurality of scripts are automatically determined. The determination is based on the plurality of clinical goals and their respective priority. Each script is configured to, when executed, adapt one or more optimization objectives and/or adapt weightings for optimization and/or provide and/or adapt an optimization structure.

Each clinical goal may have associated therewith one or more scripts that target at meeting the clinical goal. These scripts may involve optimization methods. Executing a script manipulates the optimization problem subsequently solved to obtain the dose distribution.

For example, scripts may change an optimization objective, for example, may change constraints and/or target values for the dose. Scripts may also change weightings for optimization, for example, may reduce or increase penalties when certain constraints or target values are not met. Scripts may also provide and/or adapt an optimization structure. As an example, helper structures in the form of helper volumes may be created that are not part of the main target region and constraints and/or target values and/or weightings may be provided for each helper volume. Such optimization structures may aid in meeting some optimization objectives in the main target region, potentially to the detriment of other regions, without immediately leading to penalizations. Such optimization structures may also aid in protecting sensitive regions outside of the main target regions, e.g., by having higher penalties than in other regions for certain violations of objectives.

Moreover, execution of each script may comprise an optimization that yields a dose distribution. Where the script created and/or changed an optimization structure, objectives, and/or weightings, said created and/or changed optimization structure, objectives, and weightings, respectively, may be used for the optimization. Where no changes are made to previously determined optimization structures, objectives, and/or weightings, said previously determined optimization structure, objectives, and/or weightings may be used for the optimization. Thus, a mix of added, changed, and previously determined optimization structures, objectives, and/or weightings may serve as input for the optimization.

A script may not only perform one of the above-described steps, but may perform any combination thereof. Example scripts are also provided further below.

It can be understood from the above that the selection of suitable scripts depends on the clinical goals. Moreover, the order in which to suitably perform the scripts depends on the priorities of said clinical goals. For example, creating a protective optimization structure as outlined above, may be performed prior to other steps in case protection of a certain region is a higher priority goal.

It may in some cases be helpful to lock certain results, such that execution of subsequent scripts does not change said results. This will be explained in more detail below. As an example, once the protective optimization structure has been created, the presence of the structure and at least some of the associated constraints may be locked. This may avoid that lower-priority optimization goals counteract, as part of some trade-off, the protective effect provided by the optimization structure.

It is noted that the automatic selection of scripts and the order in which they are performed may take different forms. The selection may be rule-based or may be based on a look-up that allows for retrieving a certain selection and order for any given selection and prioritization of clinical goals, depending on the case at hand.

In step S14, a dose distribution is generated. This step comprises at least the step S14a of executing the subset of scripts in the determined order. Step S14a comprises, for each of the scripts, step S14a-1 of obtaining at least one of an optimization structure, optimization objectives, and weightings for optimization, and step S14a-2 of performing an optimization based thereon.

When it comes to step S14a, reference is made to the explanations of the preceding steps, which illustrate that the execution of the scripts will create and/or adapt optimization structures, optimization objectives and/or weightings. Accordingly, since creating and/or adapting is performed for each script, the optimization structures, optimization objectives and/or weightings may be changed iteratively. Executing all scripts successfully may result in a final optimization structure, final optimization objectives, and final weightings.

The optimization structure obtained for each script, including the final optimization structure, may comprise one or more optimization (sub-)structures, e.g., a main target region and/or one or more helper structures, e.g., helper volumes.

Regarding step S14a-2, once an optimization structure, potentially including helper structures, optimization objectives, and weightings have been determined, an optimization will be performed based thereon. The optimization will yield a dose distribution.

Steps S14a-1 and S14a-2 are performed for each of the scripts, such that the dose distribution, obtained by performing an optimization based on the respective optimization structure, objectives, and weightings, may be iteratively adapted.

For the sake of completeness, it is noted that creating and/or adapting the optimization structure, objectives, and/or weightings may involve solving optimization problems as well. By keeping certain optimizations separate from each other (in the form of separate scripts) and performing them in a predetermined order, a better control over the trade-offs that are made and, particularly, that are not made, is possible.

FIG. 1 also shows optional step S15. This step illustrates that optionally at least part of the results obtained by executing a script may be locked, such that the locked part cannot be modified by execution of subsequent scripts. Locking may be performed multiple times at different stages of the method, which is, however, not shown individually in FIG. 1, but collectively summarized as step S15.

FIG. 1 also shows optional step S16 of, upon determining that a dose distribution cannot be determined without changing a locked optimization objective and/or a locked weighting for optimization and/or a locked optimization structure, e.g., locked from the beginning or after executing a script, aborting the generating of the dose distribution and optionally initiating informing a user of a failure to determine a dose distribution and/or of potential reasons for and/or solutions to a failure to determine a dose distribution.

All steps of the method may be performed without requiring user input, particularly, fully automatically. Optionally, all steps may be performed fully automatically unless a failure occurs, in which case a user may be prompted to select or confirm next steps.

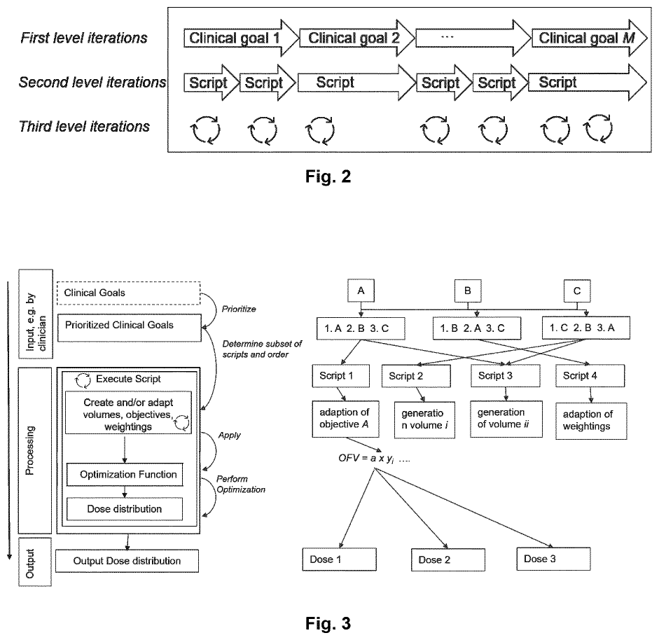

FIG. 2 schematically illustrates the iterative and hierarchical nature of a method according to the present disclosure.

This method of the present disclosure may take a prioritized list of clinical goals as input, for example two or three, and aim at achieving the goals successively, in order of priority, particularly while maintaining previously achieved clinical goals. This first optimization level, also referred to as first iteration level, comprises a number of iterations corresponding to the number of clinical goals, i.e., there is one iteration per clinical goal.

On a second, lower optimization level or iteration level, the method of the present disclosure provides multiple scripts being performed in a specific order, each script corresponding to an iteration of the second iteration level. Each script may define a scheme of optimization interaction steps. These steps may change optimization structures, optimization objectives (e.g., dose and/or volume objectives) and/or optimization weightings, e.g. objective weights. One or more scripts may be associated with one clinical goal, i.e., with one iteration on the first optimization level.

It is noted that scripts may be obtained based on development using expert knowledge and explorative testing in advance. Some such scripts will be outlined in more detail below.

On a third, even lower optimization level or iteration level, as part of executing scripts, optimizations are iteratively performed, wherein, for example, each script may entail performing one or more such optimizations.

The second and third levels may interact as follows, in an example. A script may aim at adapting dose constraints and/or optimization structures. On the third level, objectives like dose constraints and optimization structures, may be adapted iteratively. As an example, for each second level iteration, an optimization problem may be formulated with an objective function and the objective function may be minimized. Such an objective function penalizes deviation from objectives. The second and third level may, thus, allow for exploration of the search space feasible, for representing clinical objectives in a feasible manner (it may be difficult to provide an objective function representative of complex objectives), and/or for preventing the low-level optimization from getting stuck in local minima.

FIG. 3 schematically illustrates a method of the present disclosure.

In this example clinical constraints and clinical goals A, B, C and their priorities are (at least in part) determined based on a user input, e.g., a clinician's input.

On the left, goals A, B, and C are prioritized in descending order. For this prioritization, the Scripts and 3 are automatically selected and carried out in this order.

In the middle, goals B, A, and C are prioritized in descending order. For this prioritization, the Script 4 is automatically selected and carried out.

On the right, goals C, B, and A are prioritized in descending order. Based on the prioritization, scripts are selected. For this prioritization, the Scripts 2 and 3 are automatically selected and carried out in this order.

The order of carrying out the scripts is not illustrated in the Figure for simplicity purposes.

In this example, script 1 adapts an objective related to clinical goal A, script 2 generates an optimization structure, referred to as “volume i” in the Figure, and script 3 generates another optimization structure, referred to as “volume ii” in the Figure. Script 4 adapts weightings.

Thus, the scripts create and/or adapt an optimization structure, objectives, and weightings, respectively.

Execution of each script also involves an optimization to obtain a dose distribution, An optimization function OFV may be used and optimization may be performed taking into account the optimization structure(s), objectives, and/or weightings obtained when executing the script, thereby obtaining a dose distribution after execution of each script. Different dose distributions (“Dose 1”, “Dose 2”, “Dose 3”) may be obtained for the three different prioritization scenarios. After execution of the last script, a final dose distribution is obtained as an output.

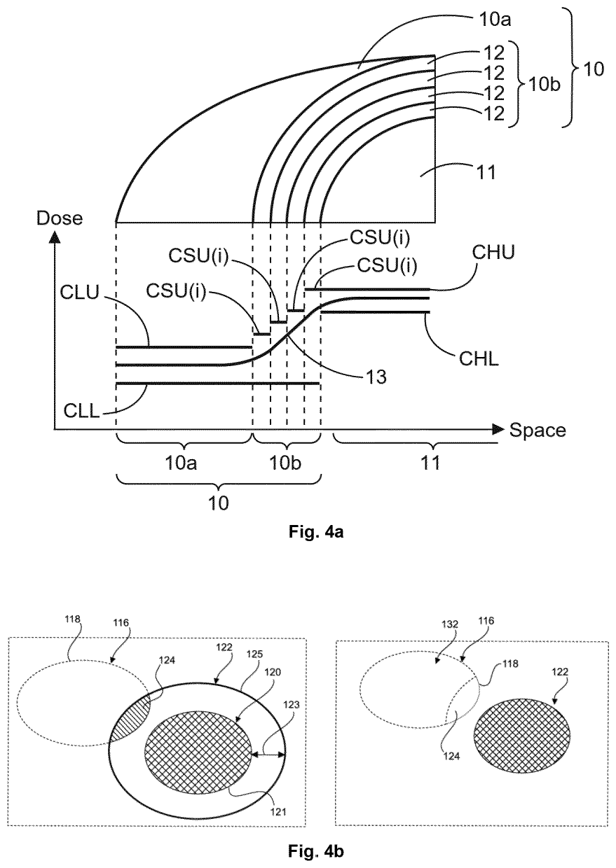

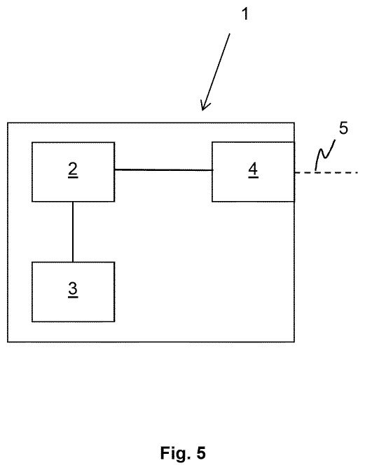

In the following, two examples for scripts will be provided in more detail making reference to FIGS. 4a and 4b.

A first script may concern creation of helper structures, specifically, a transition region around a main target region, also referred to as high dose target volume in the following.

The script may comprise the step of determining, based on a predetermined lower dose constraint C_{HL} of a high dose target volume and an upper dose constraint C_{LU} of a low dose target volume surrounding and adjacent to the high dose target volume, a transition region target thickness Iₜ of a transition region comprised in the low dose target volume and adjacent to the high dose target volume. The script may further comprise the step of determining, based on the thickness Iₜ a number n of shells to be created so as to form the transition region and creating n shells Sᵢ, where i=0 to n-1. The script may further comprise determining, for each of the shells, a shell-specific upper dose constraint C_{SU}(i) based at least on the lower dose constraint C_{HL} of the high dose target volume and the upper dose constraint C_{LU} of the low dose target volume, wherein the upper dose constraint of at least one of the shells is higher than the upper dose constraint C_{LU} of the low dose target volume and wherein the shell-specific upper dose constraint C_{SU}(i) increases from the outermost shell Sₙ₋₁ to the innermost shell S₀. The script may further comprise the step of generating, by means of an optimization algorithm, a dose distribution, the optimization algorithm constrained by the predetermined lower dose constraint C_{HL} in the high dose target volume, the upper dose constraint C_{LU} in the low dose target volume except for the transition region, and the respective shell-specific upper dose constraint C_{SU}(i) for each of the shells in the transition region.

In other words, using this script, a transition region, also referred to as a build-up region or expansion volume, with modified dose constraints may be automatically created at an interface between the high dose target volume, which may be the main target volume described above and may also be referred to as a boost target volume, and the low dose target volume. The transition region may be constituted by shells, which may be seen as helper volumes or helper objects. The transition region and/or its shells may be seen as examples for optimization structures as mentioned throughout the present disclosure. To some controlled extent, the transition region expands the high dose target volume into the low dose target volume.

The above-described script facilitates the optimization in effectively shaping the dose distribution at the interface between the low dose and high dose target volume, particularly such that the constraints are kept.

In FIG. 4a, this is illustrated schematically. The low dose target volume 10, high dose target volume 11, and several shells 12 are shown. A transition target region 10b is formed by the shells. The remaining volume of the low dose target volume (outside of the target region) is labelled 10a.

Moreover, the predetermined lower dose constraint C_{HL} of the high dose target volume and an, for example predetermined, upper dose constraint C_{LU} of the low dose target volume are shown. In addition, an, for example predetermined, upper dose constraint C_{HU} of the high dose target volume and a predetermined lower dose constraint C_{LL} of the low dose target volume are shown. In addition, the respective shell-specific upper dose constraint C_{SU}(i) for each of the shells in the transition region is shown. It can be seen that the constraint increases towards the high dose target volume and, particularly, in the present disclosure, the innermost shell has an upper dose constraint that equals the upper dose constraint of the high dose target volume.

An exemplary dose distribution 13 as obtained when performing an optimization based on the above optimization structure and objectives is also shown.

Details on the concept of such a transition region as a helper structure are provided in the international patent application PCT/EP2022/063077 (“METHOD FOR DETERMINING A TREATMENT PLAN INCLUDING A DOSE DISTRIBUTION”). In particular, the script outlined above may carry out the method as shown in FIG. 1 thereof and described in the context of said FIG. 1.

A second exemplary script may concern adapting the main target region to take into account organs at risk. When carrying out the second script, an optimized planning target volume is obtained. The script is illustrated in FIG. 4b.

The script may perform the step of providing an initial coverage volume 118 for a planning target volume 116 to be irradiated in an irradiation treatment with a prescribed dose. The script may also perform the step of providing at least one constraint for an organ at risk 120, the at least one constraint being indicative of an allowed dose deposited in at least a part or partial volume of the organ at risk. In a further step, an organ dose deposited in said at least part or partial volume of the organ at risk is calculated, when applying an initial irradiation treatment plan and/or according to the initial irradiation treatment plan. The script further comprises the step of determining an amount of violation of the at least one constraint based on comparing the at least one constraint and the calculated organ dose. The script further comprises the step of calculating a reduction coverage volume for the planning target volume based on the determined amount of violation. The script further comprises the step of generating a virtual planning object 122 by changing, e.g., increasing or decreasing, a volume of the organ at risk, such that an overlap region 124 of the virtual planning object with the planning target volume corresponds to the reduction coverage volume. This step may comprise determining the overlap region. The script may further comprise the step of generating an optimized planning target volume 132 to be irradiated during the irradiation treatment based on and/or by reducing the initial coverage volume of the planning target volume based on and/or by removing at least a part of said overlap region from the planning target volume. With the above-described script, an optimum trade-off between a biologically effective dose deposited in at least a partial volume of the planning target volume, e.g. corresponding to the optimized planning target volume, and the at least one constraint and/or the sparing of the organ at risk may be found.

Also shown in the Figure, for sake of illustration, are a radius 123 and an outer surface, contour, perimeter and/or circumference 125 of the virtual planning object 122.

Details on the concept of obtaining optimized planning target volumes taking into account an organ at risk are provided in the international patent application PCT/EP2018/055207 (“IRRADIATION TREATMENT PLANNING BASED ON TARGET COVERAGE REDUCTION”). In particular, the script outlined above may carry out the method as shown in FIG. 1 thereof and described in the context of said FIG. 1.

Other scripts are conceivable, as outlined in the claims and throughout the description in detail.

In FIG. 4, a schematic illustration of a data processing system 1 according to the present disclosure is shown. The data processing system may comprise at least processing means 2 and storage means 3, which may comprise temporary memory, e.g., RAM, and/or permanent memory, e.g., ROM. Moreover, optionally the processing system may comprise one or more communication interfaces 4 for receiving and transmitting data via one or more data connections 5. For example, the processing system may comprise one or more computers.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A computer-implemented method for determining a treatment plan for a radiotherapy treatment including a dose distribution, the method comprising the steps of:
determining a plurality of clinical goals, the plurality of clinical goals associated with a dose distribution;
determining, based at least in part on a user input, a priority for each of the clinical goals;
automatically determining a subset and order of scripts among a plurality of scripts, based on the plurality of clinical goals and their respective priority, each script configured to, when executed, adapt one or more optimization objectives and/or adapt weightings for optimization and/or provide and/or adapt an optimization structure; and
generating a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

2. The method of claim 1, wherein at least part of an optimization objective and/or a weighting for optimization and/or an optimization structure yielded by executing a script or a sequence of scripts is locked, such that the locked part cannot be modified by execution of subsequent scripts.

3. The method of claim 1, wherein, upon determining that a dose distribution cannot be determined without changing a locked optimization objective and/or a locked weighting for optimization and/or a locked optimization structure, which are locked from the beginning or after executing a script, the method aborts the generating of the dose distribution and optionally initiates informing a user of a failure to determine a dose distribution and/or of potential reasons for and/or solutions to a failure to determine a dose distribution.

4. The method of claim 1, wherein each script is executed constrained by at least part of the results of previously executed scripts, wherein an optimization performed when executing the script is constrained by the at least part of the results of previously performed scripts.

5. The method of claim 1, wherein the generating of the dose distribution is an iterative process comprising at least a first level of iterations, wherein each iteration is associated with exactly one of the clinical goals and the iterations are performed in the order of the priority of the clinical goals.

6. The method of claim 5, wherein the iterative process comprises a second level of iterations associated with the scripts, wherein each iteration of the second level of iterations is associated with exactly one of the scripts, wherein each iteration of the first level of iterations is associated with one or more iterations of the second level of iterations.

7. The method of claim 6, wherein each iteration of the second level of iterations comprises performing an optimization process or a sequence of optimization processes.

8. The method of claim 1, wherein the generating of the dose distribution is a hierarchical process, wherein each iteration of the first level of iterations ranks higher than the subsequent iterations of the first level of iterations and/or wherein, among the iterations of the second level of iterations associated with one of the iterations of the first level of iterations, each iteration ranks higher than the subsequent iterations.

9. The method of claim 1, wherein the clinical goals comprise at least one of high dose coverage of a main target region for irradiation, steep dose gradient towards regions adjacent to a/the main target region for irradiation, low upper dose limits in regions adjacent to a/the main target region for irradiation, high lower dose limits in a/the main target region for irradiation, high dose homogeneity, particularly in a/the main target region for irradiation, dose upper limits for a region defined as risk structure.

10. The method of any of claim 1,
wherein the optimization objectives comprise one or more constraints, for one or more regions, at least one of a smallest allowed dose value, a highest allowed dose value, a lowest allowed dose gradient, a lowest allowed dose homogeneity value, a highest allowed temporal dose fluctuation value, a target dose value, a target dose gradient value, a target dose homogeneity value, a target temporal dose fluctuation value; and/or
wherein the optimization structure comprises at least a main target region for irradiation and optionally comprises helper structures, around the main target region, wherein irradiation is not required but may be permitted to meet optimization objectives, and/or helper structures defining a the region defined as risk structure.

11. The method of claim 1, wherein execution of scripts may comprise at least one of:
adapting a the main target region for irradiation,
creating helper structures, around a the main target region for irradiation,
adapting helper structures, around a/the main target region for irradiation,
adapting constraints within a/the main target region for irradiation and/or within helper structures, around a the main target region for irradiation,
creating and/or adapting one or more helper structures defining a/the region defined as risk structure, and/or
adapting constraints within one or more helper structures defining a/the region defined as risk structure.

12. The method of claim 1, wherein the steps do not require user intervention.

13. A system comprising:
at least one processor having associated instructions which, when executed by at least one processor, causes the at least one processor to:
determine a plurality of clinical goals, the plurality of clinical goals associated with a dose distribution;
determine based at least in part on a user input, a priority for each of the clinical goals;
automatically determine a subset and order of scripts among a plurality of scripts. based on the plurality of clinical goals and their respective priority, each script configured to, when executed, adapt one or more optimization objectives and/or adapt weightings for optimization and/or provide and/or adapt an optimization structure; and
generate a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

14. A non-transitory computer readable medium comprising instructions which, when executed by at least one processor cause the at least one processor to:
determine a plurality of clinical goals, the plurality of clinical goals associated with a dose distribution:
determine based at least in part on a user input, a priority for each of the clinical goals;
automatically determine a subset and order of scripts among a plurality of scripts. based on the plurality of clinical goals and their respective priority each script configured to, when executed, adapt one or more optimization objectives and/or adapt weightings for optimization and/or provide and/or adapt an optimization structure; and
generate a dose distribution, wherein the generating of the dose distribution comprises executing the subset of scripts in the determined order, wherein executing the subset of scripts comprises, for each of the scripts, obtaining at least one of an optimization structure, optimization objectives, and weightings for optimization, and performing an optimization based thereon.

15. (canceled)
